(19) United States
(12) Patent Application Publication
(10) Pub. No.: US 2026/0232950 A1
(43) Pub. Date: Aug. 13, 2026

(54) VEHICLE CONTROL SYSTEM AND VEHICLE CONTROL METHOD

(71) Applicants: HYUNDAI MOTOR COMPANY, Seoul (KR); Kia Corporation, Seoul (KR)

(72) Inventors: Guo SHEN, Yantai (CN); Zhongquan SUN, Yantai (CN); Yuzhao WEI, Yantai (CN)

(21) Appl. No.: 19/534,882

(22) Filed: Feb. 10, 2026

(30) Foreign Application Priority Data
Feb. 10, 2025 (CN) 202510144229.0

(51) Int. Cl. A61M 21/00; B60H 1/00; B60Q 3/70; G06F 3/14; G06F 3/16; G07C 5/02

(52) U.S. Cl. CPC A61M 21/00; B60H 1/00764; B60Q 3/70; G07C 5/02; A61M 2021/0022; A61M 2021/0027; A61M 2021/005; G06F 3/14; G06F 3/165

(57) ABSTRACT

A vehicle control system and a vehicle control method are disclosed. The system may include: an information collection portion configured to collect driving information of a vehicle; an operation execution portion configured to provide two or more types of sensory information among a tactile sense, an auditory sense, and a visual sense corresponding to a movement state of the vehicle to an occupant of the vehicle; and a control unit configured to predict a subsequent movement state of the vehicle based on driving information of the vehicle, collected by the information collection portion, and control the operation execution portion to provide the two or more types of sensory information to the occupant.

### CROSS-REFERENCE TO RELATED APPLICATION

This application claims priority to and the benefit of Chinese Patent Application No. 202510144229.0 filed with the Chinese National Intellectual Property Administration on Feb. 10, 2025, the entire contents of which are incorporated herein by reference.

### TECHNICAL FIELD

The present disclosure relates to the vehicle control technology field, and more particularly, the present disclosure relates to a vehicle control system and a vehicle control method that can effectively prevent motion sickness.

### BACKGROUND

The matters described in this Background section are only for enhancement of understanding of the background of the disclosure, and should not be taken as acknowledgment that they correspond to prior art already known to those skilled in the art.

Motion sickness is a common and uncomfortable condition, which particularly occurs frequently when riding in a vehicle, and a main cause thereof may be explained by sensory conflict theory. The theory shows that uncomfortable symptoms such as vertigo, nausea, or vomiting may occur when information on body movement conditions transmitted from different sensory organs conflicts with each other or does not match a movement pattern expected by the brain.

Specifically, the vestibular (cochlear) organ of the human ear is like a triaxial sensor. When a vehicle is driven, the vestibular organ transmits movement information such as rotational and linear acceleration to the brain, which is prone to causing a motion sickness phenomenon by conflicting with visual static information of an occupant sitting inside the vehicle (particularly in a back seat). In particular, with the development of vehicle technology, the acceleration performance of vehicles has improved and noise has been reduced, and due to this, a difference between acceleration and deceleration experience felt by the vestibular organ and a quiet and relatively static environment inside the vehicle may have increased, thereby causing motion sickness to occur more easily.

To solve the motion sickness problem, a moving environment of a vehicle may be detected according to a driving situation of the vehicle by a sensor on a mobile phone or a tablet, and visual movement information may be transmitted to the brain by displaying the detected moving environment on a display screen of the mobile phone or the tablet. Accordingly, conflicts between the visual sense and the vestibular organ may be reduced.

However, since the focus is on a single-view sensory stimulus, only single-view motion information is transmitted to the brain when riding in a vehicle, and thus the brain's psychological expectation regarding a driving state of the vehicle may be insufficient, and thereby it may be difficult to effectively alleviate the motion sickness phenomenon. In addition, only after movement of the vehicle has changed may the driving state of the vehicle be detected by the sensor on the mobile phone or the tablet and transmitted to the brain. Therefore, the movement information transmitted to the brain may lag the movement information felt by the vestibular organ, such that the anti-motion sickness effect is reduced.

### SUMMARY

An example of the present disclosure is to provide a vehicle control system and a vehicle control method that may effectively prevent motion sickness.

According to the present disclosure, an apparatus of a vehicle may comprise a sensor configured to obtain driving information of the vehicle, a processor, and a memory storing instructions that, when executed by the processor, cause the apparatus, during a driving operation of the vehicle, to predict, based on the driving information, a subsequent movement state of the vehicle and to output, to an occupant of the vehicle via a user interface of the vehicle, at least two types of sensory information corresponding to the predicted subsequent movement state of the vehicle, wherein each type of the at least two types of sensory information corresponds to a respective one of a tactile sense, an auditory sense, or a visual sense.

In the apparatus, the driving information of the vehicle may comprise a distance between the vehicle and a preceding object and a relative speed of the vehicle to the preceding object, and the instructions, when executed by the processor, may cause the apparatus to predict, based on the distance and the relative speed, the subsequent movement state of the vehicle. In the apparatus, the driving information of the vehicle may further comprise at least one of a speed of the vehicle, a steering angle of the vehicle, a position of a brake pedal of the vehicle, or a position of an accelerator pedal of the vehicle, and the instructions, when executed by the processor, may cause the apparatus to predict the subsequent movement state of the vehicle further based on at least one of the speed, the steering angle, the position of the brake pedal, or the position of the accelerator pedal.

The apparatus may further comprise at least one of a fan system mounted on the vehicle, a speaker mounted on the vehicle, a mood light mounted on the vehicle, or a display screen mounted on the vehicle, and the instructions, when executed by the processor, may cause the apparatus to control at least two of the fan system, the speaker, the mood light, and the display screen and, based on the control of the at least two, to output to the occupant via the user interface, the speaker, the mood light, and the display screen, the at least two types of sensory information corresponding to the predicted subsequent movement state of the vehicle. In the apparatus, the display screen may be mounted on a rear row of the vehicle and connected to the apparatus via wireless communication.

The apparatus may further comprise a personal terminal connected with the apparatus via wireless communication, and the instructions, when executed by the processor, may cause the apparatus to control a speaker mounted on the personal terminal or a display screen mounted on the personal terminal and, based on the control of the speaker or the display screen of the personal terminal, to output to the occupant via the user interface the at least two types of sensory information corresponding to the predicted subsequent movement state of the vehicle. In the apparatus, the instructions, when executed by the processor, may cause the apparatus to predict, based on the driving information, an acceleration or deceleration of the vehicle and to adjust an airflow of the fan system to an airflow corresponding to the predicted acceleration or deceleration.

In the apparatus, the instructions, when executed by the processor, may cause the apparatus to predict, based on the driving information, an acceleration or deceleration of the vehicle and to output, via the speaker, a sound corresponding to the predicted acceleration or deceleration. In the apparatus, the instructions, when executed by the processor, may cause the apparatus to predict, based on the driving information, an acceleration or deceleration of the vehicle and to control the mood light such that a lighting mode of the mood light, a color of the mood light, and a brightness of the mood light are set to those corresponding to the predicted acceleration or deceleration. In the apparatus, the instructions, when executed by the processor, may cause the apparatus to predict, based on the driving information, that the vehicle is about to turn in a direction and to control the mood light such that the mood light emits light only on a side corresponding to the direction.

In the apparatus, the instructions, when executed by the processor, may cause the apparatus to predict, based on the driving information, an acceleration or deceleration of the vehicle and to display, via the display screen, a visual compensation image, wherein the visual compensation image is sized based on the predicted acceleration or deceleration. In the apparatus, the instructions, when executed by the processor, may cause the apparatus to predict, based on the driving information, that the vehicle is about to turn in a direction and to move the visual compensation image in the direction. In the apparatus, the instructions, when executed by the processor, may cause the apparatus to superimpose the visual compensation image on an image displayed on the display screen.

According to the present disclosure, a method performed by an apparatus of a vehicle may comprise, during a driving operation of the vehicle, obtaining, via a sensor of the apparatus, driving information of the vehicle, predicting, based on the driving information of the vehicle, a subsequent movement state of the vehicle, and outputting, to an occupant of the vehicle via a user interface of the vehicle, at least two types of sensory information corresponding to the predicted subsequent movement state of the vehicle, wherein each type of the at least two types of sensory information corresponds to a respective one of a tactile sense, an auditory sense, and a visual sense.

In the method, outputting the at least two types of sensory information may comprise controlling a speaker of a personal terminal or a display screen of the personal terminal, wherein the personal terminal is communicatively coupled to the vehicle, and based on the controlling of the speaker or the display screen, outputting to the occupant via the user interface the at least two types of sensory information corresponding to the predicted subsequent movement state of the vehicle.

According to the present disclosure, a vehicle may comprise a sensor configured to obtain driving information of the vehicle, wherein the driving information comprises at least one of a speed of the vehicle, a steering angle of the vehicle, a position of an accelerator pedal of the vehicle, a position of a brake pedal of the vehicle, a distance to a preceding object, or a relative speed with respect to the preceding object, a user interface configured to output sensory feedback, and a processor circuit configured to, during an autonomous driving operation of the vehicle, predict, based on the driving information, a subsequent movement state of the vehicle, wherein the subsequent movement state comprises at least one of acceleration, deceleration, or turning, and to cause the vehicle to output, to an occupant of the vehicle via the user interface, sensory feedback of at least two different sensory types corresponding to the predicted subsequent movement state, wherein each sensory type corresponds to tactile feedback, auditory feedback, or visual feedback, and wherein the sensory feedback is provided such that the occupant is notified of the predicted subsequent movement state before or during execution of the predicted subsequent movement state.

In the vehicle, the processor circuit may be configured to cause the vehicle to output the sensory feedback to the occupant by at least two of controlling a fan system of the vehicle to provide the tactile feedback, controlling a speaker of the vehicle to provide the auditory feedback, and controlling at least one of a light of the vehicle or a display screen of the vehicle to provide the visual feedback.

Therefore, according to the example of the present disclosure, it has excellent advantages in terms of both enhancing a multi-sensory coordination effect and economic feasibility and has broad applicability.

In addition, effects that may be obtained or are expected due to the example of the present disclosure will be directly or implicitly disclosed in a detailed description of the example of the present disclosure. That is, various effects expected according to the example of the present disclosure will be disclosed within the detailed description to be described later.

### BRIEF DESCRIPTION

Hereinafter, an example of the present disclosure will be described in detail with reference to the drawings. The drawings are only for illustrative purposes and are not intended to limit the example. In the drawings, like reference numerals denote like components.

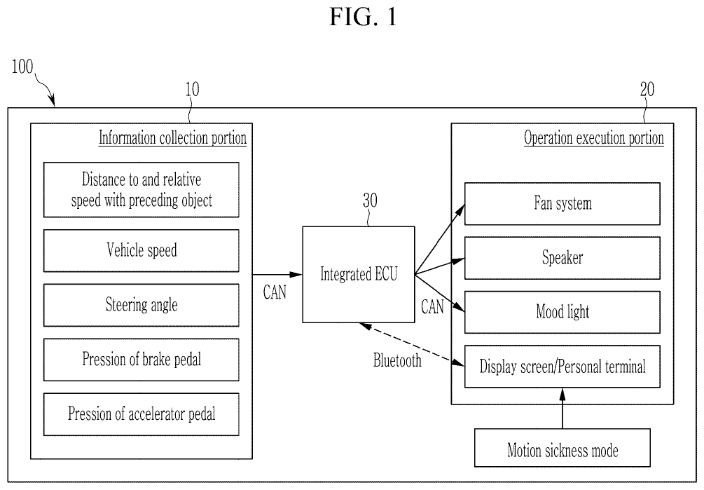

FIG. 1 shows an example of a vehicle control system.

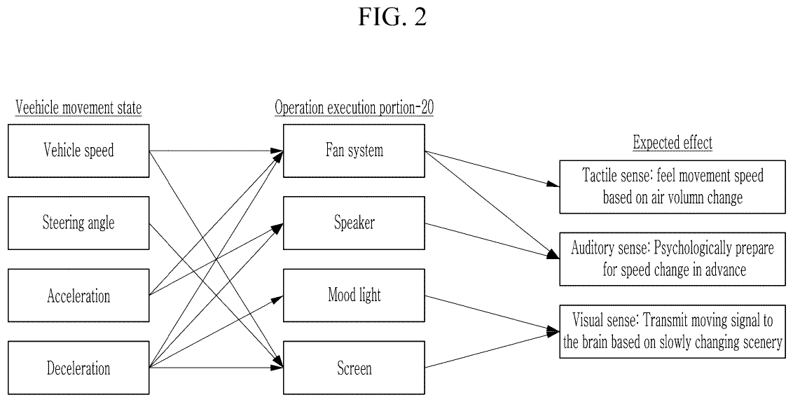

FIG. 2 shows an example of a multi-sensory coordination movement experience provided by a vehicle control system.

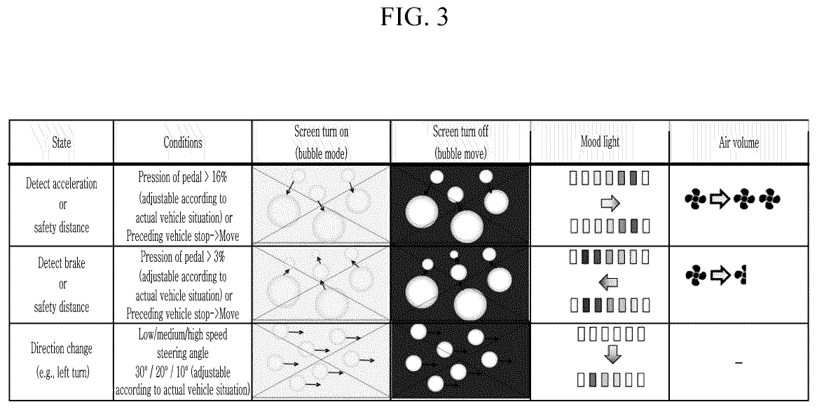

FIG. 3 shows an example of a multi-sensory coordination of the vehicle control system.

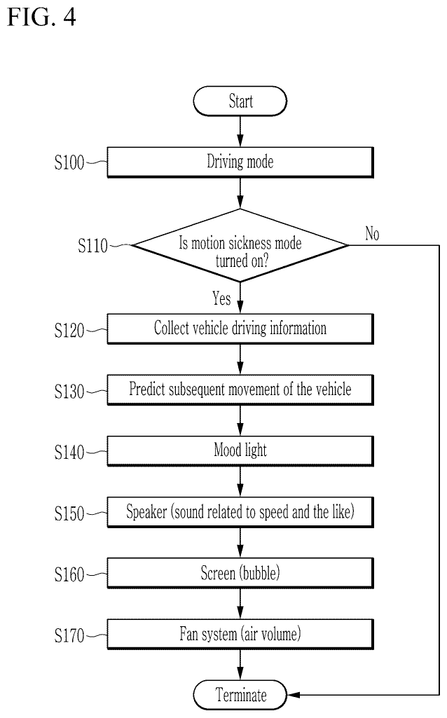

FIG. 4 shows an example of a vehicle control method.

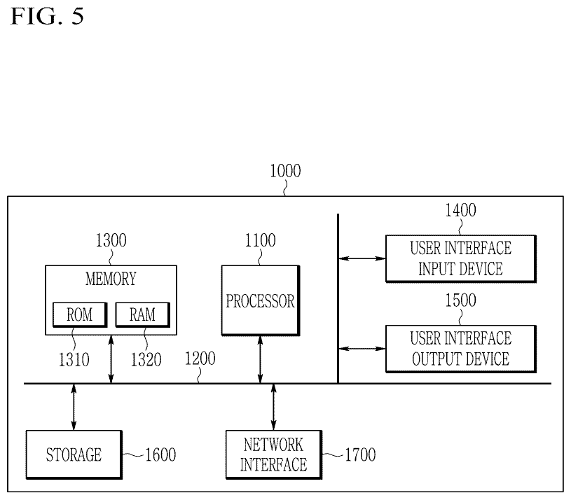

FIG. 5 shows an example computing system.

### DETAILED DESCRIPTION

Hereinafter, an example of the present disclosure will be described in detail with reference to the accompanying drawings, wherein the same or similar components may be assigned the same reference numerals, and redundant descriptions thereof may be omitted. In describing the example disclosed in the present specification, if it is determined that a detailed description of a related known art may obscure the gist of the example disclosed in the present specification, the detailed description thereof may be omitted.

The terms used in the present specification are for the purpose of describing various examples only and are not intended to limit the example of the present disclosure. A singular expression includes a plural expression unless the context clearly indicates otherwise. In the present specification, “include,” “including,” “have,” “having,” or other variations thereof are intended to specifically describe the presence of the stated features, numbers, steps, operations, components, parts, or combinations thereof, but it should be understood that they do not preclude the presence or addition of one or more other features, numbers, steps, operations, components, parts, or combinations thereof.

For purposes of this application and the claims, using the exemplary phrase “at least one of: A; B; or C” or “at least one of A, B, or C,” the phrase means “at least one A, or at least one B, or at least one C, or any combination of at least one A, at least one B, and at least one C. Further, exemplary phrases, such as “A, B, or C”, “at least one of A, B, and C”, “at least one of A, B, or C”, etc. as used herein may mean each listed item or all combinations of the listed items. For example, “at least one of A or B” may refer to (1) at least one A; (2) at least one B; or (3) at least one A and at least one B.

Additionally, it is to be understood that one or more of the following methods or examples thereof may be executed by at least one controller. The term “controller” may refer to a hardware device including a memory and a processor. The memory is configured to store program instructions, and the processor is specially programmed to execute the program instructions to perform one or more processes described in more detail below. The controller may control the operation of units, modules, parts, devices, or the like, as described herein. Furthermore, it is to be understood that the following methods may be executed by a device including a controller along with one or more other components, as would be recognized by a person skilled in the art.

In addition, a controller according to the example of the present disclosure may be implemented as a non-transitory computer-readable recording medium including executable program instructions that are executed by a processor. Examples of computer-readable recording media include, but are not limited to, a read-only memory (ROM), a random-access memory (RAM), a compact disc (CD)-ROM, magnetic tapes, floppy disks, flash drives, smart cards, and optical data storage devices. The computer-readable recording medium may also be distributed across a computer network such that program instructions may be stored and executed in a distributed manner, such as, for example, a telematics server or a controller area network (CAN).

The term “module,” “portion,” or “unit” used in the specification means a software and/or hardware component, and the “module,” “portion,” or “unit” performs certain operations/functions/roles. However, the “module,” “portion,” or “unit” is not construed as being limited to software or hardware. The “module,” “portion,” or “unit” may be configured to be in an addressable storage medium or to execute one or more processors. Therefore, as an example, the “module,” “portion,” or “unit” may include at least one of components such as software components, object-oriented software components, class components, and task components, processes, functions, attributes, procedures, sub-routines, segments of program codes, drivers, firmware, micro-codes, circuits, data, databases, data structures, tables, arrays, or variables. Functions provided in the components, “module,” “portion,” or “unit” may be combined into a smaller number of components, “module,” “portion,” or “unit” or further divided into additional components, “module,” “portion,” or “unit”.

In the present disclosure, the “module,” “portion,” or “unit” may be realized as a processor and a memory. The “processor” should be widely construed to include a general-purpose processor, a central processing unit (CPU), a microprocessor, a digital signal processor (DSP), a microcontroller, a state machine, or the like. In some environments, the “processor” may refer to an application-specific integrated circuit (ASIC), a programmable logic device (PLD), or a field-programmable gate array (FPGA), and the like. For example, the “processor” may refer to a combination of processing devices such as a combination of a DSP and a microprocessor, a combination of a plurality of microprocessors, a combination of one or more microprocessors combined with a DSP core, or any other such combination. Moreover, the “memory” should be widely construed to include any electronic component capable of storing electronic information. The “memory” may refer to various types of processor-readable medium such as a random access memory (RAM), a read only memory (ROM), a non-volatile random access memory (NVRAM), a programmable read only memory (PROM), an erasable programmable read only memory (EPROM), an electrically erasable programmable read only memory (EEPROM), a flash memory, a magnetic or optical data storage device, and registers. When the processor can read information from a memory and/or record the information in the memory, the memory may be in a state of electronic communication with a processor. Memory integrated into a processor is in a state of electronic communication with the processor.

The one or more features described herein may be provided as a computer program stored in a computer-readable recording medium to be executed on a computer. The medium may either continuously store a computer-executable program or temporarily store the program for execution or download. Furthermore, the medium may be a variety of recording or storage means in the form of a single hardware device or multiple combined hardware devices and is not limited to media directly connected to some computer system but may also be distributed across a network. Examples of such media include magnetic media such as a hard disk, a floppy disk, or a magnetic tape, optical recording media such as a CD-ROM or a DVD, magneto-optical media such as a floptical disk, and a ROM, RAM, or flash memory, among others, configured to store program instructions. Additional examples of such media include media or storage media that are managed by an app store that distributes applications or by various other sites or servers that provide or distribute software.

In a hardware implementation, processing units used for performing the techniques may be implemented within one or more ASICs, DSPs, digital signal processing devices, programmable logic devices, field-programmable gate arrays, processors, controllers, microcontrollers, microprocessors, electronic devices, or computers or combinations thereof designed to perform the functions described in the present disclosure.

An automation level of an autonomous driving vehicle may be classified as follows, according to the American Society of Automotive Engineers (SAE). At autonomous driving level 0, the SAE classification standard may correspond to “no automation,” in which an autonomous driving system is temporarily involved in emergency situations (e.g., automatic emergency braking) and/or provides warnings only (e.g., blind spot warning, lane departure warning, etc.), and a driver is expected to operate the vehicle. At autonomous driving level 1, the SAE classification standard may correspond to “driver assistance,” in which the system performs some driving functions (e.g., steering, acceleration, brake, lane centering, adaptive cruise control, etc.) while the driver operates the vehicle in a normal operation section, and the driver is expected to determine an operation state and/or timing of the system, perform other driving functions, and cope with (e.g., resolve) emergency situations. At autonomous driving level 2, the SAE classification standard may correspond to “partial automation,” in which the system performs steering, acceleration, and/or braking under the supervision of the driver, and the driver is expected to determine an operation state and/or timing of the system, perform other driving functions, and cope with (e.g., resolve) emergency situations. At autonomous driving level 3, the SAE classification standard may correspond to “conditional automation,” in which the system drives the vehicle (e.g., performs driving functions such as steering, acceleration, and/or braking) under limited conditions but transfer driving control to the driver when the required conditions are not met, and the driver is expected to determine an operation state and/or timing of the system, and take over control in emergency situations but do not otherwise operate the vehicle (e.g., steer, accelerate, and/or brake). At autonomous driving level 4, the SAE classification standard may correspond to “high automation,” in which the system performs all driving functions, and the driver is expected to take control of the vehicle only in emergency situations. At autonomous driving level 5, the SAE classification standard may correspond to “full automation,” in which the system performs full driving functions without any aid from the driver including in emergency situations, and the driver is not expected to perform any driving functions other than determining the operating state of the system. Although the present disclosure may apply the SAE classification standard for autonomous driving classification, other classification methods and/or algorithms may be used in one or more configurations described herein.

One or more features associated with autonomous driving control may activate the feature of predictive multi-sensory synchronization of vehicle motion to reduce motion sickness (e.g., based on an autonomous driving classification, a selected autonomous driving level, or an autonomous mode activation). For example, during autonomous acceleration, deceleration, or turning, the vehicle may automatically coordinate airflow, sound output, ambient lighting, and display content to anticipate vehicle motion and reduce motion sickness, while vehicle control may include autonomous driving control, braking control, acceleration control, steering control, or acceleration change-rate control, etc.

One or more auxiliary devices (e.g., an engine brake, an exhaust brake, a hydraulic retarder, an electric retarder, or a regenerative brake, etc.) may also be controlled, for example, in association with the feature of predictive multi-sensory synchronization of vehicle motion to reduce motion sickness described herein. For example, when the vehicle predicts a deceleration event and engages regenerative braking or an engine brake, the vehicle may coordinate airflow reduction, sound cues, and visual or lighting cues to synchronize occupant perception with the predicted deceleration and thereby reduce motion sickness. One or more communication devices (e.g., a modem, a network adapter, a radio transceiver, or an antenna communicating via Ethernet, Wi-Fi, Bluetooth, LTE, 5G NR, or V 2X, etc.) may be controlled to support the feature of predictive multi-sensory synchronization of vehicle motion to reduce motion sickness. For example, vehicle-to-vehicle or vehicle-to-infrastructure information may be received to predict upcoming acceleration, deceleration, or turns and to trigger corresponding multi-sensory outputs before the vehicle motion occurs. Minimum risk maneuver (MRM) operation(s) may be controlled in association with the feature of predictive multi-sensory synchronization of vehicle motion to reduce motion sickness. For example, during an MRM involving controlled deceleration or lane positioning, the vehicle may coordinate visual, auditory, and tactile cues (e.g., reduced airflow, calming audio, or simplified visual cues) to align occupant perception with the maneuver while the vehicle is brought to a minimum-risk state.

Biased driving operation(s) may be controlled in association with the feature of predictive multi-sensory synchronization of vehicle motion to reduce motion sickness. For example, when a vehicle performs biased lane positioning or a biased lane change, the vehicle may synchronize ambient lighting, display motion cues, or airflow direction with the biased lateral movement to help the occupant anticipate and adapt to the non-centered driving trajectory.

One or more sensors (e.g., camera, RADAR, LiDAR, IMU, steering angle sensor, pedal position sensor, yaw rate sensor, or vehicle speed sensor, etc.) may be used to control the feature of predictive multi-sensory synchronization of vehicle motion to reduce motion sickness. For example, sensor-detected changes in speed, steering, or relative distance to surrounding objects may be used to predict upcoming vehicle motion and to trigger coordinated tactile, auditory, and visual outputs during autonomous driving operations such as acceleration, deceleration, or turning. An autonomous driving level and/or autonomous driving activation or deactivation may be controlled in association with the feature of predictive multi-sensory synchronization of vehicle motion to reduce motion sickness. For example, when a higher autonomous driving level is activated, the vehicle may automatically enable enhanced multi-sensory synchronization during acceleration or turning, while at a lower autonomous driving level or upon deactivation, the feature may be reduced or disabled to match driver attentiveness requirements or display usage limitations.

According to the present disclosure, a vehicle control system and method are provided to help reduce motion sickness of a vehicle occupant by aligning what the occupant feels, sees, and hears with how the vehicle is about to move. Motion sickness often occurs because a person's inner ear senses acceleration, braking, or turning, while the eyes perceive a still interior, causing confusion in the brain. To address this, driving information such as vehicle speed, steering angle, pedal operation, and distance and relative speed to a preceding vehicle is collected and used to predict an upcoming movement state of the vehicle, such as acceleration, deceleration, or turning. Before or as the movement occurs, the system provides two or more coordinated sensory cues to the occupant (e.g., increasing or decreasing airflow from a fan, outputting wind or engine-related sounds, adjusting ambient lighting, or displaying a simple moving visual image on a screen) that correspond to the predicted vehicle motion. By allowing the occupant to sense the vehicle's movement in advance through multiple senses rather than a single visual cue, the brain may more easily anticipate and adapt to the motion, thereby reducing discomfort and improving ride comfort, while using sensors and devices commonly available in conventional vehicles.

Hereinafter, a vehicle control system and a vehicle control method that can prevent motion sickness according to various examples of the present disclosure will be described with reference to the accompanying drawings.

FIG. 1 shows an example of a vehicle control system 100. As shown in FIG. 1, the vehicle control system 100 includes an information collection portion 10, an operation execution portion 20, and an integrated ECU (control unit) 30.

The information collection portion 10 collects driving information of a vehicle—for example, a distance between the vehicle and a preceding object (e.g., a preceding vehicle, a motorcycle, a bicycle, or a pedestrian, etc.) through a radar and a camera mounted on a front side of the vehicle, relative speed, and the like. In addition, the information collection portion 10 may collect information such as a current vehicle speed, a steering angle (rotation angle of the steering wheel), a position of a brake pedal, a position of an accelerator pedal, and the like (e.g., pedal depression ratio, steering rate, yaw rate, or longitudinal acceleration, etc.) by an on-board sensor.

The operation execution portion 20 may be configured to provide an occupant of the vehicle with two or more types of sensory information among a tactile sense, an auditory sense, and a visual sense corresponding to a movement state of the vehicle. The operation execution portion 20 may include, for example, a conventional on-board fan system (e.g., an air conditioning system, a ventilation fan, or a climate control blower, etc.), a speaker (e.g., a cabin speaker, a headrest speaker, or a surround speaker, etc.), a mood light (e.g., an ambient LED strip, a dashboard light, or a door-panel light, etc.), and a display screen. The display screen may be, for example, a display screen in a rear seat of the vehicle (e.g., a rear-seat entertainment display, a headrest-mounted display, or a ceiling-mounted display, etc.). In addition, the operation execution portion 20 may further include a personal terminal (e.g., a mobile phone, a tablet, a wearable device, or a smart display, etc.) carried by the occupant. The operation execution portion 20 operates the fan system, the speaker, the mood light, and the display screen or a display screen of the personal terminal and a speaker of the personal terminal and provides the vehicle's occupant with two or more types of information among the tactile sense, the auditory sense, and the visual sense corresponding to the movement state of the vehicle.

The integrated ECU 30 predicts a subsequent movement state of the vehicle based on driving information of the vehicle collected by the information collection portion 10. For example, the integrated ECU 30 predicts the subsequent movement state of the vehicle based on the distance between the vehicle and the preceding object (mostly, the preceding vehicle) and the relative speed with the preceding object (e.g., closing speed, increasing distance, or stop-and-go behavior, etc.). In addition, the integrated ECU 30 predicts the subsequent movement state of the vehicle based on the steering angle of the vehicle, the position of the brake pedal, and the position of the accelerator pedal (e.g., steering input exceeding a threshold, brake pedal depression, or accelerator pedal release, etc.). Thereafter, the integrated ECU 30 controls the operation execution portion 20 to provide the vehicle's occupant with two or more types of sensory information among the tactile sense, the auditory sense, and the visual sense corresponding to the movement state of the vehicle.

The information collection portion 10 is connected to the integrated ECU 30 through, for example, a CAN bus, and transmits driving information of the vehicle to the integrated ECU 30. The integrated ECU 30 is connected to the operation execution portion 20 through a wireless communication method, such as a CAN bus or Bluetooth, and transmits a control instruction to the operation execution portion 20. Here, the integrated ECU 30 is connected to the fan system, the speaker, the mood light, and the like of the operation execution portion 20 through, for example, the CAN, and connected to the display screen (specifically, the display screen in the rear seat of the vehicle) during operation of the operation execution portion 20 or the personal terminal through a wireless communication method such as Bluetooth or Wi-Fi, etc.

For example, when a motion sickness mode is set in the display screen or the personal terminal by the occupant of the vehicle and thus the motion sickness mode is turned on, the integrated ECU 30 may activate the motion sickness mode, but the example of the present disclosure is not limited thereto. For example, a driver may turn on the motion sickness mode by manipulating a center console of the vehicle (e.g., a touchscreen interface, a physical switch, or a voice command, etc.).

FIG. 2 shows an example of a multi-sensory cooperative movement experience provided by the vehicle control system 100. As shown in FIG. 2, the vehicle control system 100 configures two or more types of sensory information among the tactile, the auditory, and the visual senses corresponding to the movement state of the vehicle for the occupant when the vehicle is being driven.

Specifically, after the motion sickness mode is turned on, the vehicle control system 100 may adjust the fan system's air amount to an air amount corresponding to a predicted amount of speed change according to the movement state of the vehicle such as driving speed, acceleration, and deceleration (e.g., rapid acceleration, gentle deceleration, or stop-and-go driving, etc.), such that a change in vehicle speed can be perceived through the tactile sense of the occupant of the vehicle and the occupant can psychologically prepare for the change. Simultaneously, the vehicle control system 100 may output a sound (e.g., wind sounds of different magnitudes, engine-like sounds, or synthesized motion sounds, etc.) corresponding to the predicted amount of speed change through the speaker according to a change in speed during acceleration or deceleration such that the occupant can associate the speed with the sound, auditorily feel the movement state of the vehicle and psychologically prepare for the change. Furthermore, at the same time, the vehicle control system 100 may also set the mood light to a lighting mode, color, and brightness corresponding to the predicted amount of speed change (e.g., increasing brightness during acceleration or decreasing brightness during deceleration, etc.), or display a visual compensation image (e.g., a bubble, an expanding shape, or a moving graphic, etc.) on the rear seat display screen, and change a size of the visual compensation image corresponding to the predicted amount of speed change, or move the visual compensation image in a turning direction of the vehicle, to visually provide the movement state such as vehicle acceleration, deceleration, and direction change to the occupant of the vehicle.

FIG. 3 shows an example of the multi-sensory cooperation of the vehicle control system 100. As shown in FIG. 3, when the position of the accelerator pedal exceeds a specified value (e.g., 16%, 20%, or another calibrated threshold, etc., adjustable according to an actual situation of the vehicle), it is predicted that the vehicle is about to accelerate, and the information collection portion 10 transmits the information to the integrated ECU 30. The integrated ECU 30 controls the operation execution portion 20 to generate a bubble on the rear seat display screen, and a size of the bubble gradually increases to simulate an acceleration state of the vehicle such that the occupant can visually perceive the acceleration of the vehicle. At the same time, the mood light lights up sequentially from a front of the vehicle to a rear (e.g., in a flowing or progressive illumination pattern, etc.), providing a visual acceleration notification to the occupant, and the fan system increases the air amount, allowing the occupant to feel the vehicle speed increasing through the tactile sense.

When the position of the brake pedal exceeds a specified value (e.g., 3%, 5%, or another calibrated braking threshold, etc., adjustable according to an actual situation of the vehicle), it is predicted that the vehicle is about to decelerate or brake, and the information collection portion 10 transmits a signal to the integrated ECU 30 and the integrated ECU 30 controls the operation execution portion 20 to correspondingly adjust the mood light and the air amount of the fan system. For example, the integrated ECU 30 reduces the brightness of the mood light and the air amount of the fan system (e.g., gradually dimming the light and reducing airflow intensity, etc.) such that the occupant can feel the deceleration of the vehicle both tactilely and visually.

When a steering angle sensor of the vehicle detects that the steering angle of 30° or more at a low speed (e.g., 0 to 30 kph), 20° or more at a medium speed (e.g., 30 to 80 kph), or 10° or more at a high speed (e.g., 80 kph or more) (the parameters of the steering angle are adjustable according to an actual situation of the vehicle (e.g., vehicle size, steering ratio, or driving mode, etc.)), it is predicted that the vehicle is about to change direction. The information collection portion 10 transmits the vehicle speed and the steering angle information to the integrated ECU 30, and the integrated ECU 30 controls the operation execution portion 20. For example, when the vehicle is about to turn left, the integrated ECU 30 moves the bubble on the rear seat display screen to the right to simulate a direction of centrifugal force during a left turn of the vehicle, and at the same time, activates only a left mood light (e.g., illuminating a left-side ambient light region, etc.). These visual and atmospheric changes help the occupant to better sense the direction change state of the vehicle. Furthermore, the speaker may also output an appropriate sound notification, such as a wind sound, a synthesized motion sound, or a slight tire friction sound, etc., according to the vehicle speed and direction change situation, thereby allowing the occupant to prepare in advance audibly.

In addition, when the vehicle follows a preceding vehicle, a safe distance is detected using a radar and/or camera mounted on the vehicle—that is, the distance and relative speed between the vehicle and the preceding object (i.e., the preceding vehicle) are detected (e.g., using adaptive cruise control sensors or forward collision warning sensors, etc.). When it detected that the distance to the preceding object is gradually decreased and thus becomes smaller than a specified value, it is predicted that the vehicle is about to decelerate. When it is detected that the distance to the preceding object is gradually increased or the preceding object has started to move from a stopped state (e.g., at a traffic light or in stop-and-go traffic, etc.), it is predicted that the vehicle is about to accelerate. The information collection portion 10 transmit such information to the integrated ECU 30. The integrated ECU 30 controls the operation execution portion 20 to cause the bubble on the rear seat display to start moving, and the mood light and the air amount of the fan system also change correspondingly, notifying the occupant that the vehicle is about to decelerate or accelerate. This multi-sensory notification, which proceeds based on the detection of the distance and the relative speed with the preceding object, also allows the occupant to predict in advance a change in the movement state of the vehicle, enabling the occupant to prepare in advance tactually, auditorily, and visually, thereby reducing the possibility of motion sickness.

In addition, when the display screen is turned on (e.g., when the occupant is watching a video, a game, or multimedia content on the display screen), the bubble (visual compensation image) may be superimposed on an image being displayed on the display screen. When the display screen is turned off, the bubble (visual compensation image) may be superimposed on a main interface, a home screen, or a standby screen, etc., of the display screen.

FIG. 4 shows an example of a vehicle control method.

In a driving mode of the vehicle (S100), the driver or the occupant communicates with an electrical control system of the vehicle through a center console of the vehicle, the rear seat display screen, or the personal terminal (e.g., a touchscreen interface, a physical switch, or a voice-based input, etc.) to select whether to turn on a motion sickness mode (S110). When the driver or the occupant does not select to turn on the motion sickness mode (“No” in S110), the corresponding step is terminated. When the driver or the occupant selects to turn on the motion sickness mode (“Yes” in S110), the vehicle turns on the motion sickness mode.

When the motion sickness mode is turned on, the integrated ECU 30 detects vehicle driving information such as a distance to and a relative speed with the preceding object, the vehicle speed, the steering angle, the position of the brake pedal, and/or the position of the accelerator pedal through—for example, a front camera, a vehicle speed sensor, a GPS sensor, a steering angle sensor, a yaw rate sensor, a brake pedal pressure sensor, and/or an accelerator pedal pression sensor of the vehicle (S120) (e.g., sensors used for adaptive cruise control, stability control, or driver assistance systems, etc.).

Thereafter, the integrated ECU 30 predicts a subsequent movement state of the vehicle, such as acceleration, deceleration, direction change, and the like, based on the collected vehicle driving information (S130) (e.g., changes in pedal input, steering input, or relative distance to a preceding vehicle, etc.).

After detecting the subsequent movement state of the vehicle, sensory information providing (S140 to S170) is performed. In the sensory information providing, the integrated ECU 30 controls at least two of the fan system, the speaker, the mood light, and the display screen mounted on the vehicle to provide sensory information of two or more types among tactile, auditory, and visual senses corresponding to the predicted movement state of the vehicle (e.g., acceleration, deceleration, or turning, etc.).

For example, when the mood light is operated (S140), it is operated separately from the left/right lamps when turning to the left/right (e.g., activating only a left-side or right-side ambient light region, etc.), thereby providing visual turning information. With respect to the vehicle speed and acceleration/deceleration, the speaker auditorily provides a wind sound, an engine sound, and the like (e.g., synthesized motion sounds or speed-dependent audio cues, etc.) to the occupant (S150). Simultaneously, a bubble (the visual compensation image) is displayed on the display screen (S160), and a size of the bubble is changed to correspond to a predicted amount of change in speed (e.g., gradual expansion during acceleration or contraction during deceleration, etc.) or the bubble is moved in a turning direction of the vehicle, to visually provide a movement state, such as vehicle acceleration, deceleration, or change in direction, to the occupant. Simultaneously, the air amount of the fan system may be changed during acceleration/deceleration (S170) (e.g., increasing airflow during acceleration or decreasing airflow during deceleration, etc.) to cause the occupant to experience a tactile change.

FIG. 5 shows an example computing system (e.g., a computing device of a vehicle or any other apparatus). One or more controllers, processors, etc. described herein, such as one or more components of the vehicle, and any other components and devices disclosed herein, may be implemented by or in the computing system as shown in FIG. 5.

A computing system 1000 may include at least one processor 1100, memory 1300, a user interface input device 1400, a user interface output device 1500, a storage 1600, and a network interface 1700, which are connected with each other via a bus 1200. The processor 1100 may be a central processing unit (CPU) or a semiconductor device that processes instructions stored in the memory 1300 and/or the storage 1600. Each of the memory 1300 and the storage 1600 may include various types of volatile or nonvolatile storage media. For example, the memory 1300 may include a read-only memory (ROM) and a random-access memory (RAM).

Communication interface(s) (also referred to as communication device(s), communicator(s), communication module(s), communication unit(s), etc.), such as the network interface 1700, may allow software and/or data to be transferred between a device and one or more external devices, and/or between one or more components of a device. Communication interface(s) may include a receiver, a transmitter, a transceiver, a modem, a network interface and/or adapter (such as an Ethernet adapter), a radio transceiver, an antenna, a communication port, a Personal Computer Memory Card International Association (PCMCIA) slot and card, or the like. Software and data transferred via communication interface(s) may be in the form of signals, which may be electronic, electromagnetic, optical, infrared, or other signals capable of being received by communication interface(s). These signals may be provided to communication interface(s) via a communication path of a device, which may be implemented using, for example, a wire or cable, fiber optics, a cellular link, a radio frequency (RF) link and/or other communications channels. Communication interface(s) may communicate using one or more communication protocols, such as Ethernet, Wi-Fi, near-field communication (NFC), Infrared Data Association (IrDA), Bluetooth, Bluetooth low energy (BLE), Zigbee, Long-Term Evolution (LTE), 5G New Radio (NR), vehicle-to-everything (V2X), a controller area network (CAN), or a local interconnect network (LIN), etc.

Accordingly, the operations of the method or algorithm described in connection with example example(s) disclosed in the specification may be implemented with a hardware module, a software module, or a combination of the hardware module and the software module, which is executed by the processor 1100. The software module may reside on a storage medium (e.g., the memory 1300 and/or the storage 1600) such as RAM, a flash memory, ROM, an erasable and programmable ROM (EPROM), an electrically erasable and programmable ROM (EEPROM), a register, a hard disk drive, a removable disc, or a compact disc-ROM (CD-ROM).

The storage medium may be coupled to the processor 1100. The processor 1100 may read out information from the storage medium and may write information in the storage medium. Alternatively, the storage medium may be integrated with the processor 1100. The processor and storage medium may be implemented with an application specific integrated circuit (ASIC). The ASIC may be provided in a user terminal. Alternatively, the processor and storage medium may be implemented with separate components in the user terminal.

According to an example of the present disclosure, a vehicle control system is provided. The system may include: an information collection portion configured to collect driving information of a vehicle; an operation execution portion configured to provide two or more types of sensory information among a tactile sense, an auditory sense, and a visual sense corresponding to a movement state of the vehicle to an occupant of the vehicle; and a control unit configured to predict a subsequent movement state of the vehicle based on the driving information of the vehicle collected by the information collection portion, and control the operation execution portion to provide the two or more types of sensory information corresponding to the predicted subsequent movement state to the occupant.

The driving information of the vehicle collected by the information collection portion may include a distance and a relative speed between the vehicle and a preceding object, and the control unit may be configured to predict the subsequent movement state of the vehicle based on the distance and the relative speed.

The driving information of the vehicle collected by the information collection portion may further include a vehicle speed, a steering angle, a position of a brake pedal, or a position of an accelerator pedal of the vehicle, and the control unit may be configured to predict the subsequent movement state of the vehicle further based on the vehicle speed, the steering angle, the position of the brake pedal, or the position of the accelerator pedal of the vehicle.

The operation execution portion may include a fan system, a speaker, a mood light, or a display screen mounted on the vehicle, and the control unit may be further configured to predict the subsequent movement state of the vehicle based on the driving information of the vehicle, and controls at least two of the fan system, the speaker, the mood light, and the display screen to provide the two or more types of sensory information corresponding to the predicted subsequent movement state to the occupant.

The display screen may be mounted on a rear row of the vehicle and connected to the control unit via wireless communication.

The operation execution portion may further include a personal terminal connected with the control unit via wireless communication, and the control unit may be further configured to predict the subsequent movement state of the vehicle based on the driving information of the vehicle, and controls a speaker or a display screen mounted on the personal terminal to provide the two or more types of sensory information corresponding to the predicted movement state to the occupant.

When it is predicted that the vehicle is about to accelerate or decelerate based on the driving information, the control unit may be further configured to control the operation execution portion to adjust an air amount of the fan system to an air amount corresponding to a predicted speed change amount.

When it is predicted that the vehicle is about to accelerate or decelerate based on the driving information, the control unit may be further configured to control the operation execution operation such that the speaker outputs a sound corresponding to the predicted speed change amount.

When it is predicted that the vehicle is about to accelerate or decelerate based on the driving information, the control unit may be further configured to control the operation execution portion such that a lighting mode, a color, and a brightness of the mood light is set to those corresponding to the predicted speed change amount.

When it is predicted that the vehicle is about to turn in a direction based on the driving information, the control unit may be further configured to control the operation execution portion such that the mood light emits light only at a side corresponding to the direction in which the vehicle turns.

When it is predicted that the vehicle is about to accelerate or decelerate based on the driving information, the control unit may be further configured to control the operation execution portion such that a visual compensation image is displayed on the display screen and a size of the visual compensation image is changed corresponding to the predicted speed change amount.

When it is predicted that the vehicle is about to turn in a direction based on the driving information, the control unit may be further configured to control the operation execution portion such that the visual compensation image is moved in the turning direction.

The visual compensation image may be superimposable on an image displayed on the display screen.

According to an example of the present application, a vehicle control method is provided. The method may include: collecting driving information of a vehicle; predicting a subsequent movement state of the vehicle based on the collected driving information of the vehicle; and providing two or more types of sensory information among a tactile sense, an auditory sense, and a visual sense corresponding to the predicted subsequent movement state to the occupant of the vehicle.

The driving information of the vehicle may include the distance between the vehicle and the preceding object and the relative speed to the preceding object, and the predicting a subsequent movement state of the vehicle may further include predicting the subsequent movement state of the vehicle based on the distance and the relative speed.

The driving information of the vehicle may further include the vehicle speed, a steering angle, a position of the brake pedal, or a position of the accelerator pedal of the vehicle, and i the predicting a subsequent movement state of the vehicle may further include predicting the subsequent movement state of the vehicle based on the vehicle speed, the steering angle, the position of the brake pedal, or the position of the accelerator pedal of the vehicle.

The providing two or more types of sensory information may include controlling at least two of a fan system, a speaker, a mood light, and a display screen mounted on the vehicle to provide the two or more types of sensory information corresponding to the predicted subsequent movement state to the occupant.

The providing two or more types of sensory information may include controlling a speaker or a display screen of a personal terminal communicatively connected to the vehicle to provide the two or more types of sensory information corresponding to the predicted subsequent movement state to the occupant.

According to an example of the present disclosure, the following technical effects can be obtained.

In the field of vehicles, particularly electric vehicles, while an improvement in acceleration and deceleration performance provides a better driving experience, a perceived difference in movement speed also becomes greater, which easily causes motion sickness. According to an example of the present disclosure, a dedicated motion sickness mode is added and a multi-sensory coordination method is used and thus more comprehensive and accurate vehicle movement state information is provided to the occupant, thereby effectively alleviating the occupant's motion sickness.

According to an example of the present disclosure, when a vehicle is in different driving states such as acceleration, braking, or turning, it is possible to accurately predict major driving states such as acceleration, deceleration, turning, and the like of the vehicle, and immediately simulate corresponding environmental changes to reduce a time difference in movement information perceived by the vestibular organ and other sensory organs, thereby enhancing an anti-motion sickness effect.

In addition, the conventional art mostly responds to the motion sickness problem by focusing on a single-view solution. According to an example of the present disclosure, various sensory stimuli are innovatively used and coordinated with various bodily senses such as tactile, auditory, and visual senses, and thus it is more effective than a conventional single-visual-movement solution. In addition, the occupant's ability to perceive and adapt to vehicle movement can be improved, thereby enhancing ride comfort. For example, when the vehicle accelerates, in addition to a visual notification on a screen and a change in ambient lighting, a sound of wind and an engine sound transmitted from the speaker allow the occupant to psychologically prepare in advance through hearing, and at the same time, an increase in air amount from the fan system allows the occupant to feel a change in vehicle speed through the tactile sense. Such omnidirectional sensory stimulation can more comprehensively simulate an actual driving environment, allowing the occupant's brain to more easily adapt to the movement state of the vehicle. Therefore, compared to the conventional single-view solution, motion sickness can be more effectively reduced, and ride comfort can be enhanced.

Furthermore, according to an example of the present disclosure, cost factors in actual application are more fully considered, and an anti-motion sickness function can be realized by using conventional sensors and devices on a vehicle. It is possible to have high practicality and economic feasibility without needing to separately invest a large amount of funds or install special high-end sensors. According to an example of the present disclosure, it may be widely applied to various vehicles, and regardless of whether it is a conventional fuel-powered vehicle, an electric vehicle, a high-end vehicle model, or a general economy vehicle, technical improvements and upgrades may all be relatively easily performed to realize the anti-motion sickness function.

The example of the present disclosure may achieve the following technical effects.

In the vehicle field, particularly in electric vehicles, although an improvement in acceleration/deceleration performance provides a better driving experience, a perceived difference in driving speed also becomes larger, thereby easily causing a motion sickness phenomenon. According to an example of the present disclosure, a dedicated motion sickness mode is added and a multi-sensory cooperation method is used such that more comprehensive and accurate vehicle motion state information can be provided to an occupant, thereby effectively mitigating the occupant's motion sickness phenomenon.

According to an example of the present disclosure, when the vehicle is in different driving states such as acceleration, braking, or change in direction, major driving states of the vehicle such as acceleration/deceleration and change in direction can be accurately predicted, and a corresponding environmental change is immediately simulated, thereby reducing a time difference in movement information perceived by a vestibular organ and other sensory organs and enhancing an anti-motion sickness effect.

In addition, the prior art mostly addresses a motion sickness problem by focusing on a single-view solution. According to an example of the present disclosure, as various sensory stimuli are innovatively used and cooperate with various bodily sensations such as the tactile, the auditory, and the visual senses, it is more effective than a conventional single-view motion solution. In addition, the occupant's ability to sense and adapt to vehicle movement can be improved, thereby improving ride comfort. For example, when the vehicle accelerates, in addition to a visual notification on the screen and a change in the mood light, a wind sound and an engine sound transmitted from the speaker allow the occupant to auditorily make psychological preparations in advance, and at the same time, an increasing air amount from the fan system allows the occupant to feel a change in vehicle speed through the tactile sense. These omnidirectional sensory stimuli can more comprehensively simulate an actual driving environment, thereby allowing the occupant's brain to adapt to a vehicle movement state more easily. Therefore, compared to a conventional single-view solution, a motion sickness phenomenon can be more effectively reduced, and ride comfort can be improved.

In addition, according to an example of the present disclosure, a cost factor in an actual application is more fully considered, and an anti-motion sickness function can be realized using sensors and devices on a conventional vehicle. Without having to separately invest a large amount of funds or install special high-end sensors, high practicality and economic feasibility can be achieved. According to an example of the present disclosure, it can be widely applied to various vehicles, and regardless of whether it is a conventional fuel-powered vehicle, an electric vehicle, a high-end vehicle model, or a general economy-class vehicle, technical improvements and upgrades can all be relatively easily carried out to realize an anti-motion sickness function.

Therefore, according to an example of the present disclosure, it has excellent advantages in terms of both improving a multi-sensory cooperation effect and economic feasibility and has wide applicability.

The example of the present disclosure described herein may be implemented as a computer-readable code on a program-recorded medium. The computer-readable recording medium may include any type of recording device on which data that is read by a computer system is stored. The computer-readable recording medium may include, for example, a hard disk drive (HDD), a solid state drive (SSD), a silicon disk drive type (SDD), a read-only memory (ROM), a random access memory (RAM), a CD-ROM, a magnetic memory, a floppy disk, an optical data storage device, and the like.

Although a preferred example of the present disclosure has been described above, the present disclosure is not limited to the above example, and includes all modifications within a scope recognized as equivalent that could be easily made from the example of the present disclosure by a person having ordinary skill in the art to which the present disclosure pertains.

What is claimed is:

1. An apparatus of a vehicle, the apparatus comprising:
a sensor configured to obtain driving information of the vehicle;
a processor; and
a memory storing instructions that, when executed by the processor, cause the apparatus to:
during a driving operation of the vehicle, predict, based on the driving information, a subsequent movement state of the vehicle, and
output, to an occupant of the vehicle via a user interface of the vehicle, at least two types of sensory information corresponding to the predicted subsequent movement state of the vehicle, wherein each type of the at least two types of sensory information corresponds to a respective one of a tactile sense, an auditory sense, or a visual sense.

2. The apparatus of claim 1, wherein:
the driving information of the vehicle comprises:
a distance between the vehicle and a preceding object, and
a relative speed of the vehicle to the preceding object, and
the instructions, when executed by the processor, cause the apparatus to predict, based on the distance and the relative speed, the subsequent movement state of the vehicle.

3. The apparatus of claim 2, wherein:
the driving information of the vehicle further comprises at least one of:
a speed of the vehicle,
a steering angle of the vehicle,
a position of a brake pedal of the vehicle, or a position of an accelerator pedal of the vehicle, and
the instructions, when executed by the processor, cause the apparatus to predict the subsequent movement state of the vehicle further based on at least one of the speed, the steering angle, the position of the brake pedal, or the position of the accelerator pedal.

4. The apparatus of claim 1, further comprising:
at least one of:
a fan system mounted on the vehicle,
a speaker mounted on the vehicle,
a mood light mounted on the vehicle, or a display screen mounted on the vehicle, and
wherein the instructions, when executed by the processor, cause the apparatus to:
control at least two of the fan system, the speaker, the mood light, and the display screen,
based on the control of the at least two of the fan system, output, to the occupant via the user interface, the speaker, the mood light, and the display screen, the at least two types of sensory information corresponding to the predicted subsequent movement state of the vehicle.

5. The apparatus of claim 4, wherein:
the display screen is mounted on a rear row of the vehicle and connected to the apparatus via wireless communication.

6. The apparatus of claim 4, further comprising:
a personal terminal connected with the apparatus via wireless communication, and
wherein the instructions, when executed by the processor, cause the apparatus to:
control a speaker mounted on the personal terminal or a display screen mounted on the personal terminal, and
based on the control of the speaker mounted on the personal terminal or the control of the display screen mounted on the personal terminal, output, to the occupant via the user interface, the at least two types of sensory information corresponding to the predicted subsequent movement state of the vehicle.

7. The apparatus of claim 4, wherein the instructions, when executed by the processor, cause the apparatus to:
predict, based on the driving information, an acceleration or deceleration of the vehicle, and
adjust an airflow of the fan system to an airflow corresponding to the predicted acceleration or deceleration.

8. The apparatus of claim 4, wherein the instructions, when executed by the processor, cause the apparatus to:
predict, based on the driving information, an acceleration or deceleration of the vehicle, and
output, via the speaker, a sound corresponding to the predicted acceleration or deceleration.

9. The apparatus of claim 4, wherein the instructions, when executed by the processor, cause the apparatus to:
predict, based on the driving information, an acceleration or deceleration of the vehicle, and
control the mood light such that a lighting mode of the mood light, a color of the mood light, and a brightness of the mood light are set to those corresponding to the predicted acceleration or deceleration.

10. The apparatus of claim 9, wherein the instructions, when executed by the processor, cause the apparatus to:
predict, based on the driving information, that the vehicle is about to turn in a direction,
control the mood light such that the mood light emits light only on a side corresponding to the direction.

11. The apparatus of claim 4, wherein the instructions, when executed by the processor, cause the apparatus to:
predict, based on the driving information, an acceleration or deceleration of the vehicle, and
display, via the display screen, a visual compensation image, wherein the visual compensation image is sized based on the predicted acceleration or deceleration.

12. The apparatus of claim 11, wherein the instructions, when executed by the processor, cause the apparatus to:
predict, based on the driving information, that the vehicle is about to turn in a direction, and
move the visual compensation image in the direction.

13. The apparatus of claim 11, wherein the instructions, when executed by the processor, cause the apparatus to:
superimpose the visual compensation image on an image displayed on the display screen.

14. A method performed by an apparatus of a vehicle, the method comprising:
during a driving operation of the vehicle, obtaining, via a sensor of the apparatus, driving information of the vehicle;
predicting, based on the driving information of the vehicle, a subsequent movement state of the vehicle; and
outputting, to an occupant of the vehicle via a user interface of the vehicle, at least two types of sensory information corresponding to the predicted subsequent movement state of the vehicle, wherein each type of the at least two types of sensory information corresponds to a respective one of a tactile sense, an auditory sense, and a visual sense.

15. The method of claim 14, wherein:
the driving information of the vehicle comprises:
a distance between the vehicle and a preceding object, and
a relative speed of the vehicle to the preceding object, and
the predicting of the subsequent movement state of the vehicle comprises predicting, based on the distance and the relative speed, the subsequent movement state of the vehicle.

16. The method of claim 15, wherein:
the driving information of the vehicle further comprises at least one of:
a speed of the vehicle,
a steering angle of the vehicle,
a position of a brake pedal of the vehicle, or
a position of an accelerator pedal of the vehicle, and
the predicting of the subsequent movement state of the vehicle further comprises predicting, based on at least one of the speed, the steering angle, the position of the brake pedal, or the position of the accelerator pedal, the subsequent movement state of the vehicle.

17. The method of claim 14, wherein the outputting of the at least two types of sensory information comprises:
controlling at least two of:
a fan system mounted on the vehicle,
a speaker mounted on the vehicle,
a mood light mounted on the vehicle, and
a display screen mounted on the vehicle; and
based on the controlling of the at least two of the fan system, the speaker, the mood light, and the display screen, outputting, to the occupant via the user interface, the at least two types of sensory information corresponding to the predicted subsequent movement state of the vehicle.

18. The method of claim 14, wherein the outputting of the at least two types of sensory information comprises:
controlling a speaker of a personal terminal or a display screen of the personal terminal, wherein the personal terminal is communicatively coupled to the vehicle; and
based on the controlling of the speaker or the display screen, outputting, to the occupant via the user interface, the at least two types of sensory information corresponding to the predicted subsequent movement state of the vehicle.

19. A vehicle comprising:
a sensor configured to obtain driving information of the vehicle, wherein the driving information comprises at least one of a speed of the vehicle, a steering angle of the vehicle, a position of an accelerator pedal of the vehicle, a position of a brake pedal of the vehicle, a distance to a preceding object, or a relative speed with respect to the preceding object;
a user interface configured to output sensory feedback; and
a processor circuit configured to:
during an autonomous driving operation of the vehicle, predict, based on the driving information, a subsequent movement state of the vehicle, wherein the subsequent movement state comprises at least one of acceleration, deceleration, or turning, and
cause the vehicle to output, to an occupant of the vehicle via the user interface, sensory feedback of at least two different sensory types corresponding to the predicted subsequent movement state,
wherein each sensory type of the at least two different sensory types corresponds to tactile feedback, auditory feedback, or visual feedback, and
wherein the sensory feedback is provided such that the occupant is notified of the predicted subsequent movement state before or during execution of the predicted subsequent movement state.

20. The vehicle of claim 19, wherein the processor circuit is configured to cause the vehicle to output the sensory feedback to the occupant by at least two of:
controlling a fan system of the vehicle to provide the tactile feedback,
controlling a speaker of the vehicle to provide the auditory feedback, and
controlling at least one of a light of the vehicle or a display screen of the vehicle to provide the visual feedback.
